(19) United States
(12) Patent Application Publication
(10) Pub. No.: US 2003/0017209 A1
(43) Pub. Date: Jan. 23, 2003

(54) Breath protection microcapsules

(76) Inventors: Rita M. Parikh, Paramus, NJ (US); Lori Dee Kumar, Skillman, NJ (US)

Correspondence Address: Darryl C. Little, Attorney for Applicant, Warner Lambert Company, 201 Tabor Road, Morris Plains, NJ, 07950, US

(21) Appl. No.: 10/141,420

(22) Filed: May 8, 2002

Related U.S. Application Data
(60) Provisional application No. 60/297,275, filed on Jun. 11, 2001.

(51) Int. Cl. A61K 31/7016; A61K 9/48; A61K 9/16; A61K 9/50

(52) U.S. Cl. 424/492; 424/493; 514/23

(57) ABSTRACT

The present invention relates to oral compositions in the form of microcapsules which reduce oral bacteria and provide long lasting breath protection comprising a select mixture of essential oils and a chlorodeoxysucrose derivative.

This application claims the benefit of U.S. Provisional Patent Application No. 60/297,275, filed on Jun. 11, 2001, the entirety of which is hereby incorporated by reference as if fully set forth herein.

### TECHNICAL FIELD

The present invention relates to oral compositions in the form of microcapsules which reduce oral bacteria and provide long lasting breath protection.

### BACKGROUND OF THE INVENTION

The use of breath control compositions such as breath mints, mouthwashes, chewing gums, etc. is widespread in most of the developed countries of the world. Another form which has been used are microcapsules containing a flavorant or other breath protection agent. These executions have acceptance due not only to their usefulness away from a place to expectorate mouthwashes but also due to the fact that they can be swallowed when the user does not need any more of the actives or doesn't want the microcapsule in the mouth any longer.

Although microcapsules have been used, there is still a need for improved such products.

Thymol is a well known antiseptic agent, also known as an essential oil, which is utilized for its antimicrobial activity in a variety of mouthwash preparations. In particular, thymol can be utilized in oral hygiene compositions such as mouth rinses in sufficient quantities to provide desired beneficial therapeutic effects. LISTERINE Registered TM-brand mouthwash is a well-known antiseptic mouthwash that has been used by millions of people for over one hundred years and has been proven effective in killing microbes in the oral cavity that are responsible for plaque, gingivitis and bad breath. Thymol, together with other essential oils such as methyl salicylate, menthol and eucalyptol, are active ingredients (e.g., antimicrobial agents) in antiseptic mouth rinses such as LISTERINE Registered TM. These oils achieve their efficacy although present in small amounts. Without being restricted to any specific theory, it is now believed that the efficacy and taste of antiseptic mouthwashes such as Listerine Registered TM may be due to the dissolution and delivery kinetics of these four active ingredients.

Unfortunately, while thymol, together whether with the other above-mentioned essential oils, provides beneficial therapeutic effects, it also provides the consumer with a flavor perception that can be described as unpleasant, harsh or medicinal in taste. A welcome contribution to the art would be compositions containing thymol wherein the unpleasant, harsh or medicinal taste of thymol has been effectively masked. Such taste masked compositions would provide the consumer with a pleasant, acceptable taste.

The present inventors have found that by incorporating a chlorodeoxysucrose derivative, the unpleasant taste of the thymol is masked, leaving the consumer with a pleasant taste perception.

It is therefore an aspect of the present invention to provide improved microcapsules.

It is another aspect of the present invention to provide microcapsules which provide improved breath control and antimicrobial activity.

It is still another aspect of the present invention to provide improved methods of providing breath control and reduction in oral bacteria.

Another aspect of the present invention is to provide improved breath control and antimicrobial mircrocapsules comprising at least one essential oil in combination with a chlorodeoxysucrose derivative.

These and other aspects of the present invention will become more apparent from the detailed description which follows.

### SUMMARY OF THE INVENTION

The present invention in one of its aspects relates to microcapsules comprising shell material and core material, wherein the microcapsules contain at least one essential oil, preferably a mixture of thymol, methyl salicylate, eucalyptol and menthol, in combination with a chlorodeoxysucrose derivative. Preferably, the microcapsules of the present invention are rapidly dissolving.

All percentages and ratios used herein are by weight unless otherwise specified. Additionally, all measurements are made at 25° C. unless otherwise specified.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The term “rapidly (or fast) dissolving ” as used herein means that the microcapsule dissolves in less than about 60 seconds, preferably less than about 30 seconds, more preferably less than about 15 seconds, after placing the microcapsule in the oral cavity.

### DETAILED DESCRIPTION OF THE INVENTION

The essential as well as optional components of the capsules of the present invention are described in the following paragraphs.

### Capsule Shell Material

The capsule shells of the present invention are manufactured using conventional capsule manufacturing technology. The shell material of the microcapsules of the present invention can be any materials which are suitable for ingestion as well as retention in the oral cavity. Materials which are suitable include gelatin, polyvinyl alcohols, waxes, gums, sucrose esters, pullulan and sugar candy type materials used in cough drops and mints, for example. For a general description of gelatin and gelatin-based capsules, see Remington's Pharmaceutical Sciences. 16^{th} ed., Mack Publishing Company, Pa. (1980), page 1245 and pages 1576-1582. Additional materials and capsule manufacturing technologies can be found in U.S. Pat. Nos.2,800,458; 3,159,585; 3,533,958; 3,697,437; 3,888,689; 3,996,156; 3,965,033; 4,010,038; and 4,016,098, each of which are herein incorporated by reference in their entirety.

The shell material is used to form any of a wide variety of shapes such as spheres, oblong shapes, disks, puffed squares and cylinders. The shell thickness is preferably in the range of about 30 um to about 2 mm, preferably from about 70 um to about 110 um. If the microcapsules are spherical, the particle diameter is generally in the range of from about 2 mm to about 9 mm, preferably from about 3 mm to about 7 mm.

### Core Materials

### Essential Oils

The microcapsules of the present invention contain a core material comprising an essential oil mixture. Preferably, the core material is present as a single-phase composition. Suitable essential oils include, but are not limited to, anethole, anise oil, bay oil, bergamot oil, bitter almond oil, bubble-gum flavoring, cedar leaf oil, cinnamic aldehyde, cinnamon oil, clove oil, eucalyptol, eucalyptus oil, eugenol, lavender oil, menthol, peppermint oil, sassafras oil, spearmint oil, terpeneless spearmint oil, thyme oil, thymol, wintergreen oil (methyl salicylate) of mixtures thereof. Preferred oils include thymol, methyl salicylate, eucalyptol, menthol and mixtures thereof.

Thymol, (CH3)2CHC6H3(CH3)OH (isopropyl-m-cresol), is only slightly soluble in water but is soluble in alcohol. Methyl salicylate, (C6H4OHCOOCH3), also known as wintergreen oil, additionally provides flavoring to the mouthwash together with an antimicrobial function. Eucalyptol (C10H18O; cineol) is a terpene ether and provides a cooling, spicy taste. Menthol (CH3C6H9(C3H7)OH; hexahydrothymol) also is highly soluble in alcohol, is fairly volatile, and in addition to any antiseptic properties provides a cooling, tingling sensation.

In the microcapsules of this invention, the essential oils are used in amounts effective to provide antimicrobial activity in the oral cavity. Generally, the total amount of essential oils present in the microcapsules can be from about 1% to about 50% w/w, optionally from about 5.0% to about 45%, or, optionally, from about 10% to about 30%, or, optionally, from about 15% to about 25%.

Thymol is preferably employed in the microcapsules of this invention in amounts of from about 0.001% to about 5% w/w, and most preferably from about 0.01% to about 3% w/w. Eucalyptol is preferably employed in amounts of from about 0.001% to about 5% w/w, and most preferably from about 0.01% to about 3% w/w. Menthol is preferably employed in amounts of from about 0.1% to about 25% w/w, most preferably from about 1% to about 20% w/w, and, optionally, from about 3% to about 15% w/w. Methyl salicylate is preferably employed in amounts of from about 0.001% to about 5% w/w, and most preferably from about 0.01% to about 3% w/w.

### Components Present in Either the Shell or the Core Material

### Chlorodeoxysucrose Derivative

The microcapsules of the present invention also comprise a chlorodeoxysucrose derivative. The chlorodeoxysucrose derivatives of the invention have the general formula (I)

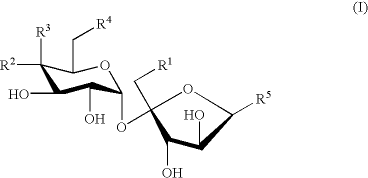

in which R¹ represents a hydroxy group or a chlorine atom; R² and R³ respectively represent a hydroxy group and a hydrogen atom, a chlorine atom and a hydrogen atom, or a hydrogen atom and a chlorine atom, the 4-position being the D-configuration; R⁴ represents a hydroxy group; or, if at least two of R¹, R², R³ and R⁵ represent chlorine atoms, R⁴ represents a hydroxy group or a chlorine atom; and R⁵ represents a hydroxy group or a chlorine atom; provided that at least one of R¹, R², R³ and R⁵ represents a chlorine atom.

The hope was that these compounds could be used to replace at least part of the sucrose in the diet, and thereby act as non-cariogenic materials.

Particular examples of compounds of the above general formula (I) are as follows (the systematic name is given first, followed by a trivial name using “galactosucrose” in those cases where an inverted 4-chloro substituent is present):

1. 1′-chloro-1′-deoxysucrose

2. 4-chloro-4-deoxy-alpha-D-galactopyranosyl-beta-D-fructofuranoside[ie 4-chloro-4-deoxygalactosucrose]

3. 4-chloro-4-deoxy-alpha-D-galactopyranosyl-1-chloro-1-deoxy-beta-D-fructofuranoside[ie 4,1′-dichloro-4,1-4,1′-dideoxygalactosucrose]

4. 1′,6′-dichloro-1′,6′-dideoxysucrose

5. 4-chloro-4-deoxy-alpha-D-galactopyranosyl-1,6-dichloro-1,6-dideoxy-beta-D-fructofuranoside[ie 4,1′,6′-trichloro-4,1′,6′-′-trideoxygalactosucrose] also known as Sucralose (McNeil Specialty Products Company, Skillman, N.J.).

6. 4,6-dichloro-4,6-dideoxy-alpha-D-galactopyranosyl-6-chloro-6-deoxy-beta-D-fructofuranoside[ie 4,6,6′-trichloro-4,6,6′-trideoxygalactosucrose]

7. 6,1′,6-trichloro-6,1′,6′-trideoxysucrose

8. 4,6-dichloro-4,6-dideoxy-alpha-D-galactopyranosyl-1,6-dichloro-1,6-dideoxy-beta-D-fructofuranoside[ie 4,6,1′,6′-tetrachloro-4,6,1′,6′-tetradeoxygalactosucrose]

9. 4,6,1′,6′-tetrachloro-4,6,1′,6′-tetradeoxysucrose.

Chlorodeoxysucrose derivatives of sucrose are known in general. They may be obtained by reacting a suitably protected sucrose with a chlorinating reagent which introduces a chlorine atom at the or each desired position. Such reagents can replace a free hydroxy group by a chlorine atom or can react with an esterified hydroxy group to introduce the chlorine. Positions requiring protection may for example be esterified or blocked with acetal or ether groups which can be easily removed after chlorination. Typical reagents include sulphuryl chloride to form the chlorosulphate ester which ester on treatment with chloride ions in turn gives the chlorodeoxysucrose derivatives. Further details of suitable preparative methods are given for example in U.S. Pat. No. 4,343,934 and 4,435,440, both of which are herein incorporated by reference in their entirety. Additional chlorodeoxysucrose derivatives can be found in U.S. Pat. No. 4,389,394, which is herein incorporated by reference in its entirety. Mixtures of the above mentioned chlorodeoxysucrose can also be used.

The chlorodeoxysucrose derivative is preferably present in the herein described microcapsules at a concentration of from about 0.001% to about 10%, more preferably from about 0.01% to about 5%, most preferably from about 0.1% to about 3%.

### OPTIONAL INGREDIENTS

### Additional Agents Suitable for Use in the Core of Capsule

Optionally and preferred for use in the microcapsules of the present invention are suitable diluents. Suitable diluents can be found in U.S. Pat. No. 4,935,243, herein incorporated by reference in its entirety. Preferred are oils such as corn, olive, rapeseed, sesame, peanut, sunflower, safflower, vegetable, or mineral. Other preferred materials include triglycerides such as capric/caprylic triglycerides (e.g., Neobee M5 [Stepan Chemical—Northfield, Ill.] and Captex 300 [Karlshams Lipid Specialties—Columbus Ohio]; distilled succinylated monoglycerides of fatty acids such as the Myverol product series (Eastman Chemicals Co.); stearate esters (Lipo) and polyethylene glycols such as PEG 400. These materials are described in further detail in U.S. Pat. Nos. 6,117,835; 6,096,338; 6,083,430; and 6,045,835, each of which are herein incorporated by reference in their entirety. These are used in an amount of from about 20% to about 80%, preferably from about 40% to about 75% of the total capsule weight.

Also optionally useful in the microcapsules of the present invention are humectants. Humectants serve to retain water on/in the surfaces of the oral cavity. Examples of suitable humectants include polyhydric alcohols selected from the group consisting of ethylene glycol, propylene glycol, dipropylene glycol, butylene glycol, hexylene glycol, polyethylene glycols, glycerin sorbitol, panthenols, urea, alkoxylated glucose derivatives, such as Glucam (RTM) E-20, hexanetriol, glucose ethers, sodium hyaluronate, soluble chitosan and mixtures thereof. Glycerin and/or sorbitol are presently preferred.

The sorbitol used in the invention is sold by the Company Roquette under the trade name Neosorb P 60 W or Neosorb p-60. The glycerin used in this invention is preferably “glycerin, USP, 99.5%”, most preferably that which is sold by Dow Chemical, Inc., Emery Industries, Inc. (under the name “Superol 99.5%”), and Procter & Gamble.

Humectants are preferably present in the microcapsules of the present invention at concentrations of from about 0.01% to about 12%, preferably from about 0.5% to about 8%, more preferably from about 1% to about 4%.

The core of the microcapsules of this invention may also contain any number of additional materials to provide additional breath freshening efficacy and/or sensory perceptions. Such agents may include quaternary ammonium salts such as pyridinium salts (e.g., cetyl pyridinium chloride), domiphen bromide, other cationic materials such as chlorhexidine salts, zinc salts and copper salts. Other agents such as phenolics, chlorhexidine, triclosan, peroxides, povidone-iodine, chlorine dioxide, neem, wild indigo, barberry, green tea, calendula, fennel, golden seal, chaparrel, chamomile, propolis, thyme, calendula as well as additional noncationic water insoluble agents are also useful herein. Such materials are disclosed in U.S. Pat. No. 5,043,154, Aug. 27, 1991, incorporated herein by reference in its entirety. Mixtures of the above mentioned breath control/antimicrobial agents may also be used. These breath control/antimicrobial agents are used in an amount of from about 0.001% to about 2%, preferably from about 0.005% to about 1% of the total core contents.

Antimalodorants useful in the present invention at levels necessary to produce the satisfactory masking of mouth malodor and include, but are not limited to, zinc salts, copper salts, chlorophyllins, apha ionones, geraniol, parsley seed and mixtures thereof.

Fluoride providing compounds may be present in the microcapsules of this invention. These compounds may be slightly water soluble or may be fully water soluble and are characterized by their ability to release fluoride ions or fluoride containing ions in water. Typical fluoride providing compounds are inorganic fluoride salts such as aminefluorides, alkali metal, alkaline earth metal, and heavy metal salts, for example, sodium fluoride, potassium fluoride, ammonium fluoride, cuprous fluoride, zinc fluoride, stannic fluoride, stannous fluoride, barium fluoride, sodium fluorozirconate, sodium monofluorophosphate, aluminum mono- and difluorophosphate, fluorinated sodium calcium pyrophosphate, acidulated monofluorophosphate and mixtures thereof.

Alkali metal, tin fluoride and monofluorophosphates such as sodium and stannous fluoride, sodium monofluorophosphate and mixtures thereof are preferred.

In the microcapsules of the present invention, the fluoride providing compound is generally present in an amount sufficient to release up to about 0.15%, preferably about 0.0005% to about 0.1% and most preferably from about 0.001% to about 0.05% fluoride by weight of the preparation.

Additionally, a variety of sweetening agents, other than (and in addition to) the chlorodeoxysucrose derivatives mentioned above, may also be included in the core or the shell of the microcapsules described herein. Suitable sweeteners may be selected from the following non-limiting list: sugars such as sucrose, glucose (corn syrup), dextrose, invert sugar, fructose, and mixtures thereof, saccharin and its various salts such as the sodium or calcium salt; cyclamic acid and its various salts such as the sodium salt; the dipeptide sweeteners such as aspartame; dihydrochalcone compounds, glycyrrhizin; Stevia Rebaudiana (Stevioside); glycyrrhizin, dipotassium glycyrrhizin, phenylalanine 1-methyl ester (Aspartame); chloro derivatives of sucrose; dihydroflavinol; hydroxyguaiacol esters; L-amino dicarboxylic acid gem-diamines; L-aminodicarboxylic acid aminoalkenoic acid ester amides; and sugar alcohols such as sorbitol, sorbitol syrup, mannitol, xylitol, and the like. Also contemplated as an additional sweetener is the nonfermentable sugar substitute (hydrogenated starch hydrolysate) which is described in U.S. Pat. No. Re. 26,959. Also contemplated is the synthetic sweetener 3,6-dihydro-6-methyl1-1-1,2,3-oxathiazin-4-one-2,2-dioxide, particularly the potassium (acesulfame-K), L-alpha-Aspartyl-N-(2,2,4,4-tatramethyl-3-thietanyl)-D-alaninamide hydrate (Alitame, a commercially available product of Pfizer, New York, N.Y.); and thaumatin (Talin).

These agents are used in an amount of from about 0.1% to about 10%, preferably from about 0.35% to about 3% of the total capsule weight. A more detailed discussion of additional as well as preferred sweetening and taste/flavor modifying materials can be found in U.S. Pat. Nos. 6,121,315 and 5,284,659, both of which are herein incorporated by reference in their entirety. Mixtures of any of the additionally disclosed sweeteners can also be used.

Particularly preferred for use in the present invention, in combination with the chlorodeoxy sucrose derivative, is acesulfame. Acesulfame is the synthetic sweetener 3,6-dihyro-6-methyl1-1-1,2,3-oxathiazin-4-one-2,2-dioxide and is, generally, incorporated into the microcapsules of the present invention as acesulfame K (Sunnett Brand Sweetener available from Hoechst Celanes, Portsmouth, Va.). Preferably the chlorodeoxysucrose derivative and acesulfame are combined at a ratio of from about 1:1 to about 9:1, more preferably from about 2:1 to about 7:3.

Vitamins such as vitamin A (retinol and carotene derivatives); vitamin B (thiamine, riboflavin, niacin, panthothenic acid, biotin, cyanocobalamin, pyridoxine, folic acid, inositol); vitamin C (ascorbic acid); vitamin D (ergocalciferol, cholecalciferol, ergosterol); vitamin E (tocopherol); vitamin K (phytonadione, menadione, phthiocol) as well as other and more specific antioxidants can also be incorporated into the microcapsules of the present invention. Suitable as well as preferred vitamins and antioxidants can be found in U.S. Pat. No. 6,238,678, herein incorporated by reference in its entirety.

The microcapsules of the present invention may also contain one or more sensory or sensate actives to act as warming or cooling signals.

When used in the present invention, sensates or sensory actives can be present at a level of from about 0.01% to about 10%, typically from about 0.1% to about 5%, and preferably from about 0.2% to about 1%. The level is selected to provide the desired level of consumer perceived sensation and can be modified as desired. Suitable sensate technologies include mannitol, inositol, physcool®, menthol, eucalyptus, 3-1-menthoxy propane-1,2-diol, N-substituted-p-menthane-3-carboxamides and acyclic carboxamides.

3-1-menthoxy propane 1,2-diol is fully described in detail in U.S. Pat. No. 4,459,425, issued Jul. 10, 1984 to Amano et. al, incorporated herein by reference in its entirety. This volatile aromatic is commercially available, being sold by Takasago Perfumery Co., Ltd., Tokyo, Japan.

The N-substituted-p-menthane-3-carboxamides are fully described in U.S. Pat. No. 4,136,163 to Watson et al., issued Jan. 23, 1979 incorporated herein by reference in its entirety. The most preferred volatile aromatic of this class is N-ethyl-p-menthane-3-carboxamide which is commercially available as WS-3 from Wilkinson Sword Limited.

Useful acyclic carboxamides are fully described in U.S. Pat. No. 4,230,688 to Rowsell et al., issued Oct. 28 1980 incorporated herein by reference in its entirety. The most preferred volatile aromatic of this class is N,2,3-trimethyl-2-isopropylbutan-amide which is commercially available as WS-23 from Wilkinson Sword Limited.

Suitable warming type sensory or sensate actives include anhydrous PEG, vanillyl alcohol n-butyl ether (TK-1000 supplied by Takasago Perfumery Co., Ltd., Tokyo, Japan), vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, iso-amylalcohol, benzyl alcohol and mixtures thereof.

Mixtures of any of the above sensory actives or sensates can also be used.

The microcapsules of the present invention may also contain sialogogues or agents that stimulate the secretion of saliva. Such agents include, but are not limited to, ascorbic acid, fumaric acid, citric acid, tartaric acid, malic acid, gluconic acid, pilocarpine, mayweed (akkal-kadha), echinacea, coleus, gentian, prickly ash, licorice, ginger, yerba santa, cardomom, monosodium glutamate and mixtures thereof.

Mucoadhesive or bioadhesives are also useful herein. Such agents include, but are not limited to, polyethylene oxide homopolymer, Carbopol®, Plasdone®, CMC, HEC, Klucel®, hydroxypropyl methylcellulose, Gantrez®, polyacrylates and mixtures thereof. These and other suitable muco- or bioadhesives along with preferred ones are detailed in U.S. Pat. Nos. 4,900,522; 5,284,659; 5,458,879; 5,989,535; 6,177,096; 6,200,604; 6,207,180; 6,210,705; 6,213,126; each of which is herein incorporated by reference in its entirety.

Water or hydroalcoholic mixtures can also present in the microcapsules of the present invention. Water comprises from about 0.1% to about 15%, preferably from about 1% to about 10%, more preferably from about 1% to about 7% of the microcapsules described herein. These amounts of water include the free water which is added, plus that amount which is introduced with other materials such as with sorbitol. The water, used in the present invention should preferably be deionized, distilled, flee of organic impurities and bacteria and substantially free of metal ions.

### Method of Manufacture

The microcapsules of the present invention can be made using a variety of conventional techniques. One method is described after the following examples.

### Industrial Applicability:

The capsules of the present invention are used by placing the capsules into the mouth and retaining them therein for a period sufficient to provide the desired effect.

The following examples further describe and demonstrate preferred embodiments within the scope of the present invention. The examples are given solely for the purposes of illustration and are not to be construed as illustrative of limitations of this invention. Many variations thereof are possible without departing from the invention's spirit and scope.

### EXAMPLES

The following compositions/capsules are representative of the present invention.

| | Ex. 1 | Ex. 2 | Ex. 3 | Ex. 4 |
|---|---|---|---|---|
| Component | % w/w | % w/w | % w/w | % w/w |
| Gelatin | 12.570 | 12.320 | 15.070 | 5.250 |
| Sorbitol | 2.060 | 2.050 | — | — |
| Acesulfame | 0.1690 | 0.1920 | — | — |
| Potassium | | | | |
| Sucralose | 0.3960 | 0.4490 | 0.641 | 0.700 |
| Glycerin | — | — | 2.04 | 2.04 |
| Water | 0.485 | 0.550 | 0.600 | 0.575 |
| Flavor | 1-10 | 1-10 | 1-10 | 1-10 |
| Thymol | 0.833 | 0.821 | 1.250 | 1.642 |
| Methyl | 0.712 | 0.700 | 1.068 | 1.400 |
| Salicylate | | | | |
| Eucalyptol | 0.781 | 0.770 | 1.172 | 1.540 |
| Menthol | 12.439 | 12.261 | 16.159 | 21.522 |
| Neobee M-5 | QS to 100% | QS to 100% | QS to 100% | QS to 100% |

The above compositions are prepared by mixing the components of the core in one container and the components of the shell(s) in another container. The shell(s) materials are heated to provide a fluid medium. The core and shell(s) materials are then pumped separately to a two or three fluid nozzle submerged in an organic carrier medium. The capsules formed are allowed to cool and stiffen. They are then denatured and separated for further handling.

In the above compositions any of a wide variety of other shell materials, breath control agents, sweeteners as well as other components may be used in place of or in combination with the components listed above.

What is claimed is:

1. A microcapsule composition, comprising a shell material and a core material, wherein said microcapsule comprises:
a.) an essential oil mixture, comprising thymol, eucalyptol, methyl salicylate and menthol; and
b.) a chlorodeoxysucrose derivative having the formula: 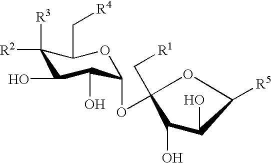
wherein R¹ represents a hydroxy group or a chlorine atom; R² and R³ respectively represent a hydroxy group and a hydrogen atom, a chlorine atom and a hydrogen atom, or a hydrogen atom and a chlorine atom, the 4-position being the D-configuration; R⁴ represents a hydroxy group; or, if at least two of R¹, R², R³ and R⁵ represent chlorine atoms, R⁴ represents a hydroxy group or a chlorine atom; and R⁵ represents a hydroxy group or a chlorine atom; provided that at least one of R¹, R^{2,} R³ and R⁵ represents a chlorine atom
and wherein the shell material is rapidly dissolving.

2. A microcapsule according to claim 1, wherein the shell material is selected from the group consisting of polyvinyl alcohol, gelatin, pullulan, waxes, gums and sugar candies.

3. A microcapsule according to claim 2, wherein the shell material is gelatin.

4. A microcapsule according to claim 2, wherein the microcapsule is in the form of a sphere or an oblong.

5. A microcapsule according to claim 4, wherein the microcapsule is in the form of spheres.

6. A microcapsule according to claim 5, wherein the microcapsule is from about 2 mm to about 9 mm in diameter and the shell wall thickness is from about 30 um to about 2 mm.

7. A microcapsule according to claim 1, further comprising an humectant.

8. A microcapsule according to claim 7, wherein the humectant is selected from the group consisting of ethylene glycol, propylene glycol, dipropylene glycol, butylene glycol, hexylene glycol, polyethylene glycols, glycerin sorbitol, panthenols, urea, alkoxylated glucose derivatives, hexanetriol, glucose ethers, sodium hyaluronate, soluble chitosan and mixtures thereof.

9. A microcapsule according to claim 1, wherein the core material, comprises:
a.) from about 0.001% to about 5% thymol;
b.) from about 0.001% to about 5% eucalyptol;
c.) from about 0.001% to about 5% methyl salicylate; and
d.) from about 0.1% to about 25% menthol.

10. A microcapsule according to claim 1, further comprising an additional sweetening component selected from the group consisting sucrose, glucose, dextrose, invert sugar, fructose, saccharin, cyclamic acid, aspartame, dihydrochalcone compounds, glycyrrhizin, Stevia Rebaudiana, dipotassium glycyrrhizin, chloro derivatives of sucrose; dihydroflavinol; hydroxyguaiacol esters, L-amino dicarboxylic acid gem-diamines, L-aminodicarboxylic acid aminoalkenoic acid ester amides, sorbitol, sorbitol syrup, mannitol, hydrogenated starch hydrolysate, acesulfame, L-alpha-Aspartyl-N-(2,2,4,4-tatramethyl-3-thietanyl)-D-alaninamide hydrate and mixtures thereof.

11. A microcapsule according to claim 1, further comprising a fluoride source selected from the group consisting of aminefluorides, alkali metal, alkaline earth metal, and heavy metal salts, for example, sodium fluoride, potassium fluoride, ammonium fluoride, cuprous fluoride, zinc fluoride, stannic fluoride, stannous fluoride, barium fluoride, sodium fluorozirconate, sodium monofluorophosphate, aluminum mono- and difluorophosphate, fluorinated sodium calcium pyrophosphate, acidulated monofluorophosphate and mixtures thereof.

12. A microcapsule according to claim 1, wherein the microcapsule dissolves in less than about 60 seconds.

13. A microcapsule according to claim 1, wherein the microcapsule dissolves in less than about 30 seconds.

14. A microcapsule according to claim 1, wherein the microcapsule dissolves in less than about 15 seconds.

15. A microcapsule according to claim 1, wherein the chlorodeoxysucrose derivative is sucralose.

16. A microcapsule composition, comprising a shell material and a core material, wherein said microcapsule comprises:
a.) an essential oil mixture, comprising thymol, eucalyptol, methyl salicylate and menthol;
b.) a chlorodeoxysucrose derivative having the formula: 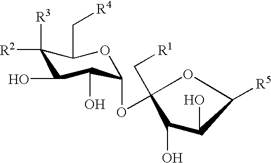
wherein R¹ represents a hydroxy group or a chlorine atom; R² and R³ respectively represent a hydroxy group and a hydrogen atom, a chlorine atom and a hydrogen atom, or a hydrogen atom and a chlorine atom, the 4-position being the D-configuration; R⁴ represents a hydroxy group; or, if at least two of R¹, R² , R³ and R⁵ represent chlorine atoms, R⁴ represents a hydroxy group or a chlorine atom; and R⁵ represents a hydroxy group or a chlorine atom; provided that at least one of R¹, R², R³ and R⁵ represents a chlorine atom; and
c.) optionally, up to about 15% water provided when water added, the water is evaporated from the microcapsule during processing such that the core material remains single-phase.

17. A microcapsule composition, comprising a shell material and a core material, wherein said microcapsule comprises:
a.) an essential oil mixture, comprising thymol, eucalyptol, methyl salicylate and menthol;
b.) a chlorodeoxysucrose derivative having the formula: 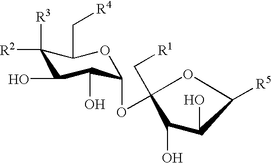
wherein R¹ represents a hydroxy group or a chlorine atom; R² and R³ respectively represent a hydroxy group and a hydrogen atom, a chlorine atom and a hydrogen atom, or a hydrogen atom and a chlorine atom, the 4-position being the D-configuration; R⁴ represents a hydroxy group; or, if at least two of R¹, R² , R³ and R⁵ represent chlorine atoms, R⁴ represents a hydroxy group or a chlorine atom; and R⁵ represents a hydroxy group or a chlorine atom; provided that at least one of R¹, R², R³ and R⁵ represents a chlorine atom; and
c.) acesulfame
wherein the ratio of the chlorodeoxysucrose derivative to acesulfame is from about 1:1 to about 9:1.

18. A microcapsule according to claim 17, wherein the ratio of the chlorodeoxysucrose derivative to acesulfame is from about 2:1 to about 7:3.

19. A microcapsule according to claim 17, further comprising an additional sweetening component selected from the group consisting sucrose, glucose, dextrose, invert sugar, fructose, saccharin, cyclamic acid, aspartame, dihydrochalcone compounds, glycyrrhizin, Stevia Rebaudiana, dipotassium glycyrrhizin, chloro derivatives of sucrose; dihydroflavinol; hydroxyguaiacol esters, L-amino dicarboxylic acid gem-diamines, L-aminodicarboxylic acid aminoalkenoic acid ester amides, sorbitol, sorbitol syrup, mannitol, hydrogenated starch hydrolysate, L-alpha-Aspartyl-N-(2,2,4,4-tatramethyl-3-thietanyl)-D-alaninamide hydrate and mixtures thereof.

20. A microcapsule composition, comprising a shell material and a core material, wherein said microcapsule comprises:
a.) an essential oil mixture, comprising thymol, eucalyptol, methyl salicylate and menthol;
b.) a chlorodeoxysucrose derivative having the formula: 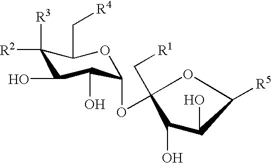
wherein R¹ represents a hydroxy group or a chlorine atom; R² and R³ respectively represent a hydroxy group and a hydrogen atom, a chlorine atom and a hydrogen atom, or a hydrogen atom and a chlorine atom, the 4-position being the D-configuration; R⁴ represents a hydroxy group; or, if at least two of R¹, R², R³ and R⁵ represent chlorine atoms, R⁴ represents a hydroxy group or a chlorine atom; and R⁵ represents a hydroxy group or a chlorine atom; provided that at least one of R¹, R², R³ and R⁵ represents a chlorine atom; and

21. A method of reducing oral bacteria or breath odor in the mouth wherein the microcapsules according to claim 1 are placed in the mouth of a human or animal in need of reducing breath odor or bacteria.
